(19) United States
(12) Patent Application Publication
(10) Pub. No.: US 2005/0003379 A1
(43) Pub. Date: Jan. 6, 2005

(54) Real happy mapping

(75) Inventors: Paul H. Dear, Cambridge (GB); Madane Thangavelu, Cambridge (GB); Alan Bankier, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 10/739,994

(22) Filed: Dec. 18, 2003

Related U.S. Application Data
(63) Continuation of application No. PCT/GB02/02780, filed on Jun. 18, 2002.

(30) Foreign Application Priority Data
Jun. 18, 2001 (GB) 0114851.9

(51) Int. Cl. C12Q 1/68

(52) U.S. Cl. 435/6

(57) ABSTRACT

The invention provides a method for identifying markers for use in mapping strategies, wherein the markers are derived from the mapping panel itself.

The present invention relates to a technique for generating markers for use in mapping strategies in mapping nucleic acids. In particular, the invention may be applied to HAPPY mapping or radiation hybrid (RH) mapping.

HAPPY mapping is a method which has been developed for linkage mapping of the genome of any organism. It was first described using single haploid sperm as a DNA source by Dear et al. in 1989 (see Dear and Cook, (1989) Nucleic Acids Res. 17:6795) and later adapted to use multiple diploid cells as a DNA source, followed by DNA dilution and aliquoting into mapping panel members, each containing ideally 0.69 haploid equivalents, using which marker linkage can be assessed; the technique has been reviewed and employed in several publications (for example, Dear and Cook, (1993) Nucleic Acids Res. 21:13-20; Piper el al., (1998) Genome Res. 8:1299-1307; and various references cited therein).

Fundamentally, HAPPY mapping involves the breaking of genomic DNA into fragments which are physically separated to provide a panel of samples, each containing an amount of DNA preferably equal to less than one haploid equivalent of the genome in question (and ideally 0.69 haploid equivalents if sampled from bulk genomic DNA). The samples are then screened for the presence of a series of markers. Markers which are located close together in the genome will cosegregate to a greater extent than markers which are more distant in the genome. By analysis of a cosegregation table obtained with a marker panel, the order and spacing of the markers in the genome can be deduced.

The main applications for HAPPY mapping include genome and gene mapping, detection of strain diversity, population analysis, epidemiology, gene expression and the demonstration of phylogenetic and taxonomic relationships.

Another technique for mapping nucleic acids which is used in the art is known as radiation hybrid (RH) mapping. In haploid RH mapping, a host (e.g. rodent)—donor (e.g. human) somatic cell hybrid containing a single human chromosome is lethally irradiated with X-rays, breaking the chromosomes into several fragments. The irradiated cells are nonviable but can be fused with a normal rodent cell line. A variety of selection methods (typically involving growth in a selective medium, survival in which requires an enzyme produced only by the irradiated cells) can be used to select hybrids arising from the fusion of an irradiated cell with an unirradiated rodent cell. Such hybrids contain, in addition to the intact set of rodent chromosomes, the fragmented chromosomes of the somatic cell hybrid; during culture, many such fragments are lost until only a subset is retained in the hybrid. Each such hybrid contains a unique set of fragments from the original human chromosome, and a clone can be typed for the presence or absence of human markers. The basic premise of RH mapping is that the closer two loci are on a chromosome, the less likely it is that radiation will induce a break between them. Thus, markers close together on a chromosome demonstrate correlated retention patterns in the hybrid clones, while loci far apart are retained nearly independently.

Haploid RH mapping has the advantage that in the absence of typing error, the number of copies of each human marker in each hybrid clone is observable. If a clone tests positive, one copy of the marker is present; if it tests negative, zero copies are present. The primary disadvantage of the haploid approach is that it is labour intensive: a separate panel of hybrids must be constructed to map each donor chromosome. To address this problem, a whole-genome RH mapping approach has been developed. This procedure involves irradiating a diploid human cell line rather than a rodent-human hybrid cell line. The advantage of the whole-genome approach is that a single set of hybrids may be used to map all human chromosomes. A disadvantage of diploid hybrids is that it is only possible to determine whether a marker is present or absent in a hybrid. If a marker is present, it is not known whether a single copy or two copies are present. Further preliminary results from some diploid hybrids suggest that the human chromosomal fragments may be retained at a lower rate in diploid hybrids than in haploid hybrids.

One of the main difficulties encountered in generating a HAPPY or RH map is the identification of suitable markers in the genome. Applications of HAPPY mapping have involved first ‘pre-amplifying’ all markers in the mapping panel simultaneously using various techniques, and then screening the pre-amplified samples by PCR for pre-defined markers, using specific primers. In an early theoretical paper describing HAPPY mapping (Dear et al., 1989), it was suggested that multiple products arising from low-stringency PCR amplification with short, arbitrary primers could serve as markers, eliminating the requirement for costly marker-specific primers. However, there remains a need for a method for generating markers for use in mapping strategies.

### SUMMARY OF THE INVENTION

The present invention provides a method for mapping a nucleic acid which comprises generating markers from a mapping panel itself. Markers obtained in such a manner have several advantages over conventional markers, and assist in fine mapping of any particular area of the nucleic acid to be mapped.

In a first aspect, therefore, the invention provides a method for nucleic acid analysis, which comprises the steps of:

a) providing a mapping panel of nucleic acid samples comprising two or more members, each member comprising a set of nucleic acid fragments;

b) pooling two or more members of the mapping panel and characterising one or more nucleic acid fragments therein to identify one or more probes; and

c) screening the mapping panel with one or more probes identified in b).

In accordance with the invention, the markers are derived from the mapping panel itself. This has a number of advantages. For instance, it is known that certain donor sequences are retained more successfully in RH panels; and also that certain methods used for pre-amplification of HAPPY mapping panels may fail to amplify certain sequences. In either case, some markers will fail to map efficiently. If all members of the mapping panel are combined, and DNA fragments from this pool are sequenced and used as markers in later mapping (using the same panel), the marker sequences will naturally be enriched in those which are successfully represented in the mapping panel; hence, the success rate for characterising markers obtained in this way will be greater than that for markers defined without reference to the mapping panel.

Moreover, it is in certain circumstances desirable to obtain markers which surround a known marker in a region which it is desired to map more finely but about which no further information may be available. In a second aspect, therefore, the invention provides a method for generating one or more markers which are linked to a known marker, comprising the steps of:

a) providing a mapping panel comprising two or more members, each member comprising a set of nucleic acid fragments;

b) identifying those members of the mapping panel which contain a first marker;

c) selecting two or more members of the mapping panel which contain the first marker and enriching said two or more members for nucleic acid fragments which are common amongst said two or more members;

d) characterising one or more fragments common amongst said two or more members to provide one or more markers which are linked to the first marker.

Any member of the mapping panel which is found to contain the first marker must contain a DNA fragment carrying this marker, and carrying adjacent DNA sequences on one or both sides thereof. However, such a panel member will also contain many other DNA fragments from parts of the genome remote from the marker. If two panel members are selected, each containing the marker, and only the DNA sequences common to both members are considered, there will be an enrichment for sequences adjacent to the marker over those remote from it. For example, if each panel member contains exactly half of a genome equivalent of fragments, then a sequence remote from the marker has only a 25% chance of being present in both members, whereas a sequence adjacent to the marker has close to a 100% chance of being present in both. If DNA sequences which are common to three, four or more panel members are considered (each of which is known to contain the marker), then there will clearly be an enrichment for sequences close to the marker over those remote from it. The relative enrichment will increase with the number of panel members considered, and decrease with increasing distance from the marker. The invention thus provides for relative enrichment for sequences showing marker linkage and is referred to as REAL HAPPY mapping.

Having enriched a pool of nucleic acid fragments for fragments which contain the marker in question, further markers which are linked thereto may be identified simply by characterising the fragments in the pool to identify marker sequences therein. For example, the fragments may be at least partially sequenced, and probes selected to be complementary to the sequences identified. Such probes will identify markers closely linked to the first marker used for the enrichment.

The mapping panel used in the invention may be any mapping panel which contains DNA fragments derived from genomic DNA or any other source which it is intended to map. Advantageously, it is a HAPPY or RH mapping panel. The panel comprises at least two members, and advantageously about 4, 8, 16, 32, 64, 96, 100, 110, 128, 256 or more members. The use of 96 members is convenient. Further members may be present as control samples.

The first marker chosen for selection of fragments may be any known marker. Advantageously, it is a marker for a region which it desired to map more finely. Any further markers obtained by the method of the invention are highly likely to be closely linked with the first marker.

Marker detection may be performed by any technique, for example by hybridisation or PCR.

The panel members found to contain the first marker are enriched for common sequences. This may be done in a variety of ways, including subtractive hybridisation in which one sample is fixed to a solid phase and denatured, whilst the second is denatured and allowed to hybridise to the first and unbound sequences are washed away. The bound sequences are common to both samples and are recovered. The same methods can be used repeatedly to isolate sequences common to more than two samples.

Markers may be identified amongst the enriched fragments by any form of characterisation which is appropriate. For example, fragments may be sequenced to identify marker sequences which may be used as probes, or which may be used as templates for complementary probes. However, it may not be necessary to sequence the markers; PCR may be used to amplify nucleic acids from the fragments which may be purified and used directly as probes, or fragments may simply be shotgun cloned and recovered inserts used as probes. However the probes are obtained, if the samples are enriched for common sequences the probes will be highly likely to detect markers closely linked to the first marker.

In a further aspect, the invention relates to a method for encoding the contents of a microtitre plate so that they can be tracked though further procedures and manipulations. The method of the invention allows microtitre plate contents to be tracked even if they are transferred or combined with samples in another plate, or loaded onto a gel.

In a first embodiment of this aspect of the invention, a solid-phase marker such as inert fluorescent microspheres (e.g., Molecular Probes A3703) is added to some of the wells in the plate; the pattern of wells thus labelled represents a unique “signature” of the plate contents. For example, the position of the markers can encode a binary number.

In a preferred embodiment, microspheres are added to a microtitre plate destined for PCR; the particles do not interfere with the reaction, and are loaded onto the gel when the samples are subsequently analysed. The ‘code’ of the microtitre plate is transferred to the gel—the wells containing the fluorescent particles light up when the gel is photographed under UV light. The particles do not migrate into the gel during electrophoresis. This overcomes a general problem in tracking samples in electrophoresis—namely that it is easy to print a barcode or similar on a microtitre plate, but difficult to transfer the coding to a gel. In an alternative embodiment, DNA is loaded into some of the unused wells of the gel to encode a binary number; however, this requires the user to read the number on the microtitre plate, and then arrange for the same number to be encoded on the gel. With the system according to the invention, the robot which sets up the PCRs can also add the fluorescent particles, and the ‘code’ is transferred to the gel upon loading, with no further intervention or opportunity for error.

The invention is moreover applicable to further embodiments. For instance, where reactions are prepared by taking one set of reagents from one plate (e.g. a set of PCR templates) and another set from another plate (e.g. a set of PCR primers), each source-plate can be encoded by spiking certain wells with the particles. Conveniently, the coding can occupy the first row of wells in one plate, and the last row in the other plate. The reaction plate (containing the contents of the two source plates) then bears both sets of encoding markers; by imaging the plate under UV illumination the codes can be seen and verified. Alternatively, the codes will be seen when the samples are analysed by gel electrophoresis.

Multiple colours can be used to provide more complex codes, or to allow two or more codes to be superimposed in the same set of wells.

### DETAILED DESCRIPTION OF THE INVENTION

Although the general techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc (as well as the complete version Current Protocols in Molecular Biology).

1. Definitions

A “mapping panel” as referred to herein is a panel of nucleic acid fragments which have been separated into separate samples, or members. Each member of the panel may consist of some fraction (typically ½ or ⅓rd) of the fragmented DNA isolated from a single haploid cell, as in Dear & Cook (1989). More generally, each member may consist of a sample of fragmented DNA prepared from two or more haploid cells or from one or more diploid cells, and ideally containing an amount of DNA equal in mass to 0.69 genomes (i.e., 0.69 haploid equivalents); this amount ensures that, assuming a Poisson distribution of sequences sampled from bulk DNA, approximately half of all markers are represented in each sample; however, amounts of DNA between about 0.05 to 2, preferably between 0.2 and 1.5, fall within the acceptable range. Use of above 2 copies is disadvantageous, but there is no rigid lower limit to the number of copies that can be used. The principle of the invention can be applied with arbitrarily low amounts of DNA per aliquot, provided that sufficient panel members are analysed to ensure that each marker sequence is represented in at least one (and preferably more than one) member.

A “marker” is a nucleic acid sequence which may be identified in linkage studies, for example by PCR or hybridisation analysis. Advantageously it is substantially unique within the genome under analysis and (if used in RH mapping) is substantially different from all sequences present in the host cell, such that identification thereof is unambiguous.

“Characterising” of nucleic acid fragments refers to any process which permits the identification of markers therein. In one embodiment, therefore, nucleic acid sequences may be characterised by sequencing. Alternatively, they may be characterised by PCR, cloning, and the like.

“Probes” are nucleic acid molecules which may be used to detect markers. For example, probes may be nucleic acids which are complementary to the markers, or primers which may be used to amplify markers.

“Enriching” refers to a procedure for increasing the proportion of a member in a population, relative to the total population. Thus, in the present invention sequences which occur in more than one sample may be enriched for by pooling the samples and eliminating those sequences which are not common to both samples, or specifically amplifying those sequences which are common.

2. HAPPY Mapping

General techniques for HAPPY mapping are well known in the art and have been extensively described in the literature. The following disclosures, which comprise detailed descriptions of HAPPY mapping, are incorporated herein by reference in their entirety: Konfortov et al., Genome Res. 2000 Nov;10(11):1737-42; Williams and Firtel, Genome Res. 2000 Nov;10(11):1658-9; Piper et al., Genome Res. 1998 Dec;8(12):1299-307; Lynch et al., Genomics. Aug. 15, 1998;52(1):17-26; Dear et al., Genomics. Mar. 1, 1998;48(2):232-41; Walter et al., Nucleic Acids Res. Sep. 25, 1993;21(19):4524-9; Dear and cook, Nucleic Acids Res. Jan 11, 1993;21(1):13-20; Dear and Cook, Nucleic Acids Res. Sep. 12, 1989;17(17):6795-807. The present invention is advantageously used for the preparation of novel markers for use in HAPPY mapping.

3. Probes

Probes as referred to herein are composed of units which are either nucleotides or nucleotide analogues. Generally speaking, a nucleotide analogue is a compound which is capable of being incorporated in a chain of nucleotide residues and which is capable of hybridising in a base-specific manner with a base of a complementary nucleic acid chain. Analogues useful in the present invention are preferably recognised by chain-extending enzymes.

A nucleotide analogue may be a modified nucleotide wherein the base is modified, for example so as to affect base-pairing properties; and/or wherein the sugar or backbone moiety is modified, for example as in the amide linked backbones of PNA; and/or wherein the phosphate moiety is modified.

Backbone-modified nucleic acids include methylphosphonates, phosphorothioates and phosphorodithioates, where both of the non-bridging oxygens are substituted with sulphur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-540 -O-phosphoroamidate. Peptide nucleic acids replace the entire phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without compromising affinity.

Modification of the heterocyclic bases preferably maintains proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Probes are preferably 7-40 residues in length overall. Usually a short probe with 15-25 residues is used, but probes with up to 30 or up to 40 residues are also useful.

Probes may incorporate a base analogue that promotes degenerate binding, by having the ability to base pair with two or three of the natural bases, or universal, by forming base pairs with each of the natural bases without discrimination. Such analogues may be used, in conjunction with conventional randomisation techniques, in the manufacture of the probes. However, it is preferred that the probes are highly sequence-specific in their binding and do not hybridise to degenerate sequences.

Nucleotides or nucleotide analogues for addition during the chain extension step may be labelled for ease of detection. Examples of suitable labels include radioisotopes, fluorescent moieties, haptens, and components of chromogenic or chemiluminescent enzyme systems.

Additionally, or alternatively, probes of defined sequence may be labelled using specific tags which allows them to be readily identified. Examples include tags having different masses, which are separable by mass spectroscopy; molecular bar-codes which may be “read” using appropriate detection instruments; combinations of fluorescent tags, which generate a specific signature emission; and the like.

The nature of the labelling will determine the best method for detection of the markers present in each sample. Where the fragments are unlabelled, or all similarly labelled, the amplified fragments are advantageously detected by gel electrophoresis, as in conventional HAPPY mapping.

However, use of specific labels allows fragments to be sorted otherwise, such as by FACS or mass spectrometry. See, for example, Griffin et al., (1997) Nature Biotechnology 15:1368. Where the labels may be made specific for each primer, individual aliquots of the sample may be sorted for the presence of specific fragments without the need for amplification.

4. Enrichment For Common Sequences

Techniques for enrichment of common sequences are known in the art. For example, subtractive hybridisation may be used, wherein one sample is immobilised and used to retain, by hybridisation, those sequences of a second sample which are common to both samples. Sequences which differ between the first and second samples are discarded. The process may then be repeated to remove non-common sequences from the first panel. In such a propcedure, the first step is the immobilisation of one panel member, hybridisation to a second (whereupon common sequences are retained), followed by recovery of these bound, common sequences by elution, usually under strongly denaturing conditions. This whole process can be repeated to further restrict the sequences to those also common to a third panel member: this can be done either by immobilising the third member, hybridising the sequences retained in the first step, and then again denaturing and eluting to recover the subset of the first panel member also present in the third member; or by immobilising the sequences retained in the first step and hybridising the third panel member thereto.

Where the method of the invention is applied to RH mapping, it is also necessary to eliminate the ‘host’ sequences which will be common to all members of the mapping panel. This can be done using subtractive hybridisation to eliminate common sequences. Alternatively, a PCR method can be used to amplify only donor sequences from a DNA sample, if a primer(s) is/are used which are complementary to a characteristic sequence present only in the donor genome. For example, if the RH panel carries human fragments in a rodent background, then subtractive hybridisation may be used to prepare a pool of fragments common to two or more members; these would include all the rodent sequences as well as a small set of human sequences. Amplification using primers complementary to human “Alu” sequence motifs then yields only human-derived sequences which lie between Alu elements.

Subtractive hybridisation techniques may be supplemented with amplification techniques to more rapidly enrich the pool for common sequences. PCR may be performed to exploit the 2nd order kinetics of self-reassociation seen in genomic DNA, whereby after an initial subtraction target sequences are enriched relative to non-target sequences and in subsequent rounds of hybridisation these target sequences more readily find their complement. Double-stranded sequences can be amplified preferentially by removing single-stranded sequence using a single-strand specific nuclease such as S1 nuclease. This leads to exponential enrichment of target sequences, using a first round of subtractive hybridisation and subsequent rounds of amplification.

5. Screening The Mapping Panel

Mapping panels may be screened according to any standard method known in the art, including gel electrophoresis and PCR. HAPPY mapping panels are screened according to standard procedures (see above) to generate linkage tables, from which the degree of linkage may be assessed. The screening of the mapping panel, and linkage analysis, may be automated.

Screening of RH panels may be performed as previously described; see, for example, Goss S. J., Harris H., “New method for mapping genes in human chromosomes” Nature. 1975. V. 225. P. 680-684; Goss S. J., Harris H., “Gene transfer by means of cell fusion. I. Statistical mapping of the human X-chromosome by analysis radiation-induced gene segregation” J. Cell Sci. 1977. V. 25. P. 17-37; Goss S. J., Harris H., “Gene transfer by means of cell fusion. II. The mapping of 8 loci on human chromosome 1 by statistical analysis of gene assortment in somatic cell hybrids” J. Cell Sci. 1977. V. 25. P. 39-57; Goss S. J., “Radiation-induced segregation of synthetic loci: a new approach to human gene mapping” Cytogenet. Cell Genet. 1976. V. 16. P. 138-141. Advantageously, RH panels are typed by PCR detection of marker sequences. Stewart, E. A. and Cox, D. R., “Radiation hybrid mapping” in “Genome Mapping: A Practical Approach” ed. Dear, P. H., IRL Press, 1997.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for nucleic acid analysis, which comprises the steps of:
a) providing a mapping panel of nucleic acid samples comprising two or more members, each member comprising a set of nucleic acid fragments;
b) pooling two or more members of the mapping panel and characterising one or more nucleic acid fragments therein to identify one or more probes; and
c) screening the mapping panel with one or more probes identified in b).

2. A method according to claim 1, wherein all members of the mapping panel are pooled.

3. A method for generating one or more markers which are linked to a known marker, comprising the steps of:
a) providing a mapping panel comprising two or more members, each member comprising a set of nucleic acid fragments;
b) identifying those members of the mapping panel which contain a first marker;
c) selecting two or more members of the mapping panel which contain the first marker and enriching said two or more members for nucleic acid fragments which are common amongst said two or more members;
d) characterising one or more fragments common amongst said two or more members to provide one or more markers which are linked to the first marker.

4. A method according to claim 1 or 3, wherein markers are characterised by sequencing.

5. A method according to claim 1 or 3, wherein the mapping panel is a HAPPY mapping panel or a RH mapping panel.

6. A method according to claim 1 or 3, wherein the mapping panel comprises 2, 4, 8, 16, 32, 64, 96, 100, 110, 128 or 256 members.

7. A method according to claim 3, wherein the nucleic acid fragments are enriched by subtractive hybridisation.
